(19) United States
(12) Patent Application Publication
(10) Pub. No.: US 2007/0129658 A1
(43) Pub. Date: Jun. 7, 2007

(54) Compression device

(76) Inventors: David M. Hampson, Cardiff (GB); Ryhs Morris, South Glamorgan (GB)

Correspondence Address: DEWITT ROSS & STEVENS S.C., 8000 EXCELSIOR DR, SUITE 401, MADISON, WI, 53717-1914, US

(21) Appl. No.: 10/596,389

(22) PCT Filed: Dec. 10, 2004
(86) PCT No.: PCT/GB04/05207

§ 371 Date: Feb. 5, 2007

(30) Foreign Application Priority Data
Dec. 12, 2003 (GB) 0328774.5

(51) Int. Cl. A61F 5/00

(52) U.S. Cl. 602/13; 602/1

(57) ABSTRACT

A compression garment (1) that wraps around a limb has a bladder (3) inflated by a pump (not shown) to apply pressure to a specific area of the limb in order to empty the veins in that limb and upon release of that pressure by deflation the bladder (3), there is increased blood flow in the arterial system. The garment (1) also warms the tissues, typically between 32 and 42 degree centigrade, the heating achieved by passing an electric current through a conductive material (5) that coverts the electrical energy into heat. The material (5) and an outer layer (6) of the garment are joined at their peripheries enclosing the bladder (3) within. The garment (1) provides all round warming of the limb and gradual compression to a part of the limb at low pressure, proven to be effective in improving arterial blood flow and more comfortable to the user.

The present invention relates to an inflatable compression device, in particular an inflatable compression device for improving arterial blood flow.

Existing inflatable compression devices for improving arterial blood flow comprise a wrap-around garment having inflatable bladders, the garment wrapped around a lower limb and the bladders inflated by a pump to apply pressure to the limb. Normally, the bladders are inflated rapidly at high pressure in order to empty the veins in the limb reducing the venous pressure, so that upon rapid deflation of the bladders, the reduced venous pressure results in an increased blood flow (hyperaemic response) in the arterial system.

Such inflatable compression devices are known to increase flow in the major blood vessels and over time, alleviate the symptoms of arterial disease, but are generally not comfortable for the user due to the use of rapid inflation at high pressures.

The present invention seeks to make improvements.

Accordingly, the present invention provides an inflatable compression device comprising a garment to encircle a limb, the garment having at least one bladder inflatable by a pump to apply pressure to a specific area of the limb covered by the garment, the garment further having means to warm substantially the whole of the limb covered by the garment. The combination of compression and warming is more efficient than systems that provide just compression alone, or warming alone. The device acts on the tissues at the site of compression, empties the veins, to promote a hyperaemic response in the arteries and so improves arterial flow. Over time, collateral flow in the diseased arteries is improved. The additional warming promotes flow to the superficial tissues by means of vasodilatation of the arterioles and capillaries. This overall warming of the area augments and compliments the effects of the local compression and the overall effect is greater than using each method alone. Heating the tissues increases blood flow to the parts where ulcers and lesions occur and in doing so, help to cure them. The compression prevents the accumulation of the products of accelerated metabolism that may be promoted by warming, and by promoting hyperaemia ensures re-perfusion of oxygenated blood to tissue. Therefore, a combination of compression and warming improves significantly the general flow of fluids in the limb, both nutritional flow into the limb via the arteries and drainage of fluids at the lesions.

Preferably, the pressure applied to the limb is low and gradual, such that the compression device of the present invention operates at much lower pressures than existing compression devices. The gradual inflation of the bladder with warming is far more comfortable and better tolerated by the users, ensuring patient compliance. We have found that heating the whole area of the limb covered by the garment gave better blood flow measurements than heating only part of the limb. Furthermore, improved arterial flow was achieved with applying pressure to only specific areas of the limb covered by the garment resulting in a simpler garment requiring only a small bladder and better patient compliance. Advantageously, this combined compression and warming acts upon both systemic flow and superficial tissues at the same time.

In a preferred embodiment, the warming means includes an electrically heated flexible material forming an inner layer of the garment. Alternatively, the warming can be achieved by heating elements attached to an inner layer of the garment. Other possibilities for the warming material include conductive polymers, carbon fibres, and foils. Preferably, the inner layer of the garment is joined together with an outer layer at their peripheries enclosing the bladder in between the layers, and more preferably both layers of the garment are vapour permeable to allow the transmission of sweat from the limb to the atmosphere.

The present invention is described by way of example below, with reference to the accompanying drawings in which:

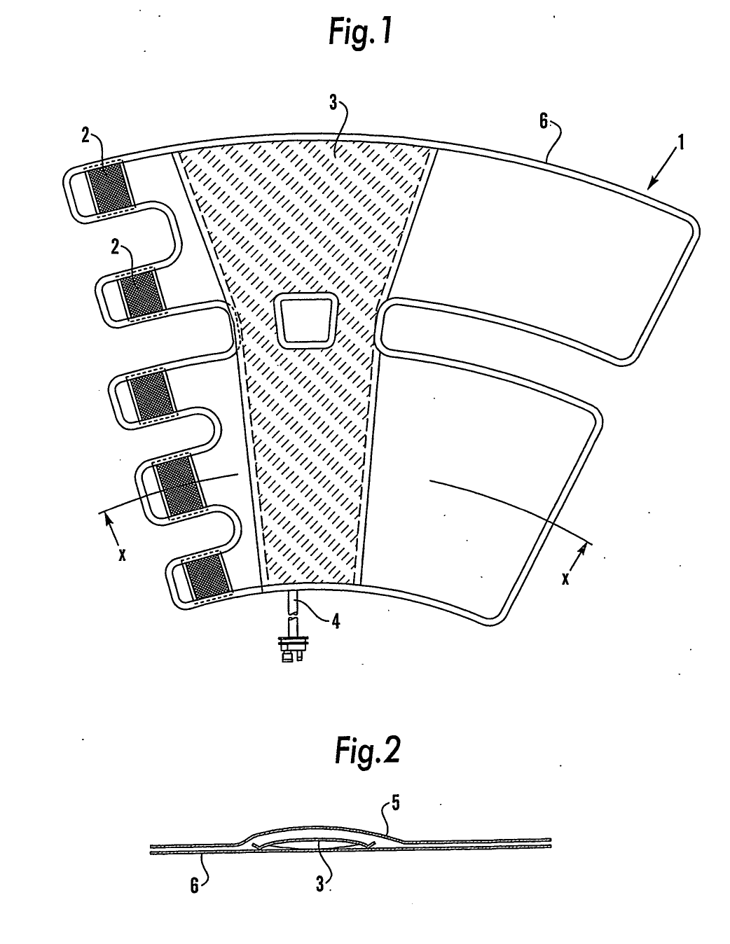

FIG. 1 is a schematic diagram of a preferred embodiment of the invention;

FIG. 2 shows a cross-section of the garment in FIG. 1 along X-X.

Referring to FIG. 1, the device consists of a compression garment 1 that encircles a lower limb and is held in place with hook fasteners 2, although other means such as a zip, or even a slip on garment can be used.

The garment 1 has an air bladder 3 to apply pressure to a part, for example, the calf of a lower limb when inflated. The bladder 3 is made from flexible plastic film welded together to form an air tight enclosure with an integral supply hose 4. The bladder 3 is smaller than the garment and can take any form sufficient to compress only a specific area of the limb covered by the garment. The bladder 3 can be inflated to a pressure and time duration appropriate to the desired therapy by means of a pump (not shown) via hose 4. The bladder 3 is attached to the outer layer 6 of the garment.

The garment 1 also includes an inner layer of thermally transmissive material 5 to apply warming to the tissues (typically between 32 and 46 degree centigrade) by passing an electrical current through the material 5. Possible materials include conductive polymers, carbon fibres, wire elements and foils. In another embodiment the inner layer 5 can be a material 5 housing electrical heating elements suitably insulated and extending the full area of the garment to provide heat to the whole circumference of the limb. The inner 5 and outer layers 6 of the garment are joined together at their peripheries enclosing the bladder 3 in between the layers. The outer layer 6 of the garment 1 is inextensible to provide effective compression of the limb upon inflation of the bladder such that as the bladder inflates it also imparts a reactive force to the encircling material which also compresses the limb.

Both the outer and inner layers 5, 6 are breathable to allow the transmission of water vapour and thereby avoid sweating of the limb.

We have found that a cycle of 60 mmHg pressure compression for 10-12 seconds, followed by a decompression for 49 seconds allowing the venous system to refill, is the most effective in improving arterial flow. We have also found that that when compared to existing systems our protocol of a low pressure of 60 mmHg for only 10-12 seconds is all that is needed to achieve a hyperaemic response and therefore increase arterial flow.

The present invention provides a compression garment that whilst applying low pressure to only part of a limb covered by the garment warms the whole area of the limb covered by the garment providing a more comfortable garment proven to improve arterial flow.

1. An inflatable compression device comprising a garment to encircle a limb, the garment having at least one bladder inflatable by a pump to apply pressure to a specific area of the limb covered by the garment, the garment further having means to warm substantially the whole of the limb covered by the garment.

2. An inflatable compression device as claimed in claim 1 wherein the warming means comprises an electrically heated flexible material.

3. An inflatable compression device as claimed in claim 1 wherein the warming means comprises heating elements.

4. An inflatable compression device as claimed in claim 1 wherein the garment comprises an inner layer of material having the warming means, the inner layer joined together with an outer layer of the garment at their peripheries enclosing the bladder in between the layers.

5. An inflatable compression device as claimed in claim 4 wherein both the inner and outer layers of the garment are vapour permeable.

6. An inflatable compression device as claimed claim 1 wherein the pressure applied to the limb is 60 mmHg over 10-12 seconds.

7. An inflatable compression device as claimed in claim 4 wherein the outer layer is formed of inextensible material.

8. An inflatable compression device as claimed in claim 4 wherein the garment includes fasteners at opposing portions of the joined peripheries of the inner and outer layers, whereby the garment may be wrapped about a limb and fastened at the fasteners to maintain the garment on the limb.

9. An inflatable compression device as claimed in claim 1 wherein:
a. the inflatable bladder is defined over only a portion of the garment,
b. the garment includes at least one noninflatable portion extending from the bladder, the noninflatable portion defining a flap which is wrappable about at least a portion of a limb.

10. An inflatable compression device as claimed in claim 8 wherein both the inflatable bladder and noninflatable portion are defined between inner and outer layers of material.

11. An inflatable compression device comprising:
a. an inextensible outer sheet;
b. an inner sheet extending at least substantially parallel to the outer sheet;
c. an inflatable bladder situated between the inner and outer sheets.

12. The inflatable compression device of claim 11 wherein the outer sheet and inner sheet are joined along their peripheries, and wherein the bladder is spaced from at least a portion of the joined peripheries.

13. The inflatable compression device of claim 12 wherein the bladder is affixed to the outer layer of the device.

14. The inflatable compression device of claim 11 further comprising heating elements on or within the inner sheet.

15. The inflatable compression device of claim 14 wherein the entirety of the inner sheet is heated.

16. The inflatable compression device of claim 11 wherein the bladder is affixed to the outer layer of the device.

17. The inflatable compression device of claim 11 further comprising a pump in communication with the bladder, the pump cyclically providing gas to and removing gas from the bladder, wherein the pump repeatedly:
a. provides gas to the bladder for up to 2/10 of a minute; and
b. subsequently removes at least some of the gas from the bladder for the remainder of the minute.

18. The inflatable compression device of claim 11 wherein the pump provides gas to the bladder at a pressure of no greater than approximately 60 mmHg.
